(19) United States
(12) Patent Application Publication
(10) Pub. No.: US 2024/0410906 A1
(43) Pub. Date: Dec. 12, 2024

(54) DHEAS ASSAY, REAGENTS FOR SAME, AND METHODS OF PRODUCTION AND USE THEREOF

(71) Applicant: Siemens Healthcare Diagnostics Inc., Tarrytown, NY (US)

(72) Inventors: Zhijian Zhao, Wilmington, DE (US); Yi Feng Zheng, Wilmington, DE (US); Andrew Rybin, Sleepy Hollow, NY (US); James Freeman, Sandy Hook, CT (US)

(73) Assignee: Siemens Healthcare Diagnostics Inc., Tarrytown, NY (US)

(21) Appl. No.: 18/700,260

(22) PCT Filed: Sep. 23, 2022
(86) PCT No.: PCT/US2022/076904

§ 371 Date: Apr. 10, 2024

Related U.S. Application Data
(60) Provisional application No. 63/262,472, filed on Oct. 13, 2021.

(51) Int. Cl. G01N 33/74; G01N 21/64; G01N 33/543; G01N 33/58

(52) U.S. Cl. CPC G01N 33/743; G01N 21/6428; G01N 33/54326; G01N 33/582; G01N 2021/6439

(57) ABSTRACT

Immunoassay reagents are disclosed that contain a dehydroepiandrosterone sulphate-fluorescein (DHEAS-FITC) conjugate or a dehydroepiandrosterone sulphate-carboxy methoxylamino-dimethyl acridinium ester (DHEAS-CMO-DMAE) conjugate. Also disclosed are immunoassay kits and devices that contain one or more of the DHEAS conjugates. Methods of making and using the DHEAS conjugates are further disclosed.

### CROSS REFERENCE TO RELATED APPLICATIONS/INCORPORATION BY REFERENCE STATEMENT

Not Applicable.

### STATEMENT REGARDING FEDERALLY SPONSORED RESEARCH OR DEVELOPMENT

Not Applicable.

### BACKGROUND

Dehydroepiandrosterone Sulphate (DHEAS) is an adrenal steroid that has the chemical structure depicted in FIG. 1, and abnormal levels of DHEAS are known in the art to be associated with many different disorders and conditions. For example (but not by way of limitation), measurement of circulating levels of DHEAS is important in investigations of abnormal hair growth (hirsutism) and balding (alopecia) in women. Also, measurement of DHEAS levels is of value in the assessment of adrenarche and delayed puberty, as plasma levels of DHEAS increase steadily from about the seventh year of life, then gradually decline after the third decade. In addition, DHEAS is often assayed in conjugation with free testosterone as an initial screen for hyperandrogenism in hirsutism, and high DHEAS levels are often encountered in polycystic ovary syndrome (PCOS). Further, levels of DHEAS greater than 700-800 μg/dL in women are suggestive of a hormone-secreting adrenal tumor.

Methods for the measurement of DHEAS are desired in order to provide a mechanism for the assessment of DHEAS levels in human patients. However, immunoassays for the detection of DHEAS have encountered issues with assay sensitivity, ambient temperature effect (ATE), and susceptibility to biotin interference.

Therefore, there is a need in the art for new and improved DHEAS assay reagents and assays, as well as methods of producing and using same to detect DHEAS levels in biological samples.

### BRIEF DESCRIPTION OF THE DRAWINGS

FIG. 1 depicts the chemical structure of dehydroepiandrosterone sulphate (DHEAS).

FIG. 2 illustrates one non-limiting embodiment of a DHEAS assay format constructed in accordance with the present disclosure.

FIG. 3 illustrates another non-limiting embodiment of a DHEAS assay format constructed in accordance with the present disclosure.

FIG. 4 provides a scheme for synthesis of one non-limiting embodiment of an assay reagent that may be utilized in accordance with the present disclosure, DHEAS-CMO-EDA-Fluorescein.

FIG. 5 provides a scheme for synthesis of another non-limiting embodiment of an assay reagent that may be utilized in accordance with the present disclosure, DHEAS-CMO-PEG3-Fluorescein.

FIG. 6 provides a scheme for synthesis of another non-limiting embodiment of an assay reagent that may be utilized in accordance with the present disclosure, DHEAS-BSA-Fluorescein.

FIG. 7 provides a scheme for synthesis of yet another non-limiting embodiment of an assay reagent that may be utilized in accordance with the present disclosure, a DHEAS-CMO-EDA-DMAE Conjugate.

FIG. 8 provides a scheme for synthesis of yet another non-limiting embodiment of an assay reagent that may be utilized in accordance with the present disclosure, a DHEAS-CMO-Z-NSP-DMAE Conjugate.

FIG. 9 graphically depicts test definition optimization of binding curves for the DHEAS assay format of FIG. 2 using the assay reagent DHEAS-CMO-EDA-Fluorescein versus a commercial assay (which uses DHEAS). Test definitions 811-833 feature varying sequences and timings of reagent additions using DHEASII format, but all show improved binding profile compared to commercial DHEAS assay.

FIG. 10 graphically depicts binding curves for the DHEAS assay format of FIG. 3 using the DHEAS-CMO-EDA-DMAE Conjugate versus a commercial assay (which uses DHEAS).

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions/devices, kits, and/or methods disclosed herein can be made and executed without undue experimentation in light of the present disclosure. While the compositions/devices, kits, and/or methods have been described in terms of particular embodiments, it will be apparent to those of skill in the art that variations may be applied to the compositions/devices, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “liquid test sample” as used herein will be understood to include any type of biological fluid sample that may be utilized in accordance with the present disclosure. Examples of biological samples that may be utilized include, but are not limited to, whole blood or any portion thereof (i.e., plasma or serum), saliva, sputum, cerebrospinal fluid (CSF), intestinal fluid, intraperitoneal fluid, cystic fluid, sweat, interstitial fluid, tears, mucus, urine, bladder wash, semen, combinations thereof, and the like. The volume of the liquid test sample utilized in accordance with the present disclosure may be (for example but not by way of limitation) from about 0.1 μl to about 100 μl.

As used herein, the term “volume” as it relates to the liquid test sample utilized in accordance with the present disclosure means from about 0.1 μl to about 100 μl, or from about 1 μl to about 75 μl, or from about 2 μl to about 60 μl, or less than or equal to about 50 μl.

The term “patient” includes human and veterinary subjects. In certain embodiments, a patient is a mammal. In certain other embodiments, the patient is a human. “Mammal” for purposes of diagnosis/treatment refers to any animal classified as a mammal, including human, domestic and farm animals, nonhuman primates, and zoo, sports, or pet animals, such as dogs, horses, cats, cows, etc.

Turning now to particular non-limiting embodiments of the inventive concept(s), the present disclosure is related to immunoassay reagents (as well as kits and devices containing same) that can be utilized in methods of determining the concentration of DHEAS in a biological sample. The immunoassay reagents of the present disclosure include novel DHEAS conjugates, and immunoassays that utilize these novel DHEAS conjugates overcome the issues of assay sensitivity, ambient temperature effect (ATE), and susceptibility to biotin interference observed in currently available DHEAS immunoassays.

Certain non-limiting embodiments of the present disclosure include an immunoassay reagent that comprises a dehydroepiandrosterone sulphate-fluorescein (DHEAS-FITC) conjugate. Any DHEAS-FITC conjugate disclosed or otherwise contemplated herein may be included in the immunoassay reagents constructed in accordance with the present disclosure. In particular non-limiting embodiments, the DHEAS-FITC conjugate comprises DHEAS-CMO-EDA-FITC (DHEAS-carboxymethoxylamino-ethyldiamine-fluorescein), DHEAS-CMO-PEG3-Fluorescein (DHEAS-carboxymethoxylamino-(polyethylene glycol)3-Fluorescein), or DHEAS-CMO-BSA-Fluorescein (DHEAS-carboxymethoxylamino-bovine serum albumin-Fluorescein).

The term “fluorescein” as used in the context of the DHEAS conjugates will be understood to refer to fluorescein alone as well as to fluorescein derivatives, such as (but not limited to) fluorescein isothiocyanate (FITC).

Certain non-limiting embodiments of the present disclosure are directed to an immunoassay reagent that comprises a dehydroepiandrosterone sulphate-carboxymethoxylamino-dimethyl acridinium ester (DHEAS-CMO-DMAE) conjugate. Any DHEAS-CMO-DMAE conjugate disclosed or otherwise contemplated herein may be included in the immunoassay reagents constructed in accordance with the present disclosure. In particular non-limiting embodiments, the DHEAS-AE conjugate comprises DHEAS-CMO-EDA-DMAE (DHEAS-carboxymethoxylamino-ethyldiamine-dimethyl acridinium ester) or DHEAS-CMO-Z-NSP-DMAE (DHEAS-carboxymethoxylamino-Z-N-sulfopropyl-dimethyl acridinium ester).

Certain non-limiting embodiments of the present disclosure are directed to kits useful for conveniently performing an immunoassay for the determination of a concentration of DHEAS. These kits include any of the DHEAS conjugates disclosed or otherwise contemplated herein above, either alone or in combination with other assay reagents disclosed or otherwise contemplated herein. In addition, the kits may further contain other component(s) and/or reagent(s) for conducting any of the particular assays described or otherwise contemplated herein. The nature of these additional reagent(s) will depend upon the particular assay format, and identification thereof is well within the skill of one of ordinary skill in the art.

In certain non-limiting embodiments of the present disclosure, the immunoassay kits include any of the DHEAS-FITC conjugates disclosed or otherwise contemplated herein, either alone or in combination with anti-fluorescein paramagnetic solid phase particles and/or an acridinium ester-labeled anti-DHEAS monoclonal antibody.

Any paramagnetic solid phase particles (PMP) known in the art or otherwise contemplated herein may be utilized in accordance with the present disclosure. The term “paramagnetic” refers to substances in which slight magnetic properties may be introduced resulting in a weak attraction to either pole of a magnet, a state that is lost upon removal from the magnetic field. Paramagnetic substances typically have unpaired “d” electrons. Paramagnetic substances include, but are not limited to, metal salts such as, for example, metal oxides, and metal halides, for example; and metallic elements, for example. The metal may be, by way of illustration and not limitation, iron, chromium, lithium, sodium, magnesium, aluminum, manganese, strontium, zirconium, molybdenum, ruthenium, rhodium, palladium, tin, samarium, europium, tungsten, platinum, and combinations thereof, for example.

In certain non-limiting embodiments, the paramagnetic particles generally have an average diameter of about 0.02 to about 100 microns, or about 0.05 to about 100 microns, or about 0.1 to about 100 microns, or about 0.5 to about 100 microns, or about 0.02 to about 50 microns, or about 0.05 to about 50 microns, or about 0.1 to about 50 microns, or about 0.5 to about 50 microns, or about 0.02 to about 20 microns, or about 0.05 to about 20 microns, or about 0.1 to about 20 microns, or about 0.5 to about 20 microns, for example. In some embodiments, the particles have an average diameter from about 0.05 microns to about 20 microns or from about 0.3 microns to about 10 microns, or about 0.3 microns to about 5 microns, for example. In some embodiments, by way of illustration and not limitation, the paramagnetic particles are iron (II) oxide particles, iron (III) oxide particles, mixtures of iron (II) oxide and iron (III) oxide particles, chromium oxide particles, and particles formed from oxides of lithium, sodium, magnesium, aluminum, manganese, strontium, zirconium, molybdenum, ruthenium, rhodium, palladium, tin, samarium, europium, tungsten, or platinum, and mixtures of two or more of the above, for example.

The paramagnetic solid phase particles may be coated or uncoated; when a coating is present, the coating may be (for example but not by way of limitation) an aldehyde coating, a polymer coating, a copolymer coating, etc.

Anti-fluorescein antibodies are well-known in the art, widely commercially available, and have been vastly studied. For example (but not by way of limitation), a few commercial sources of anti-fluorescein antibodies include those available from Abbexa Ltd (Houston, TX); Abcam (Cambridge, UK); Biorbyt Ltd (St. Louis, MO); Jackson Immuno Research Labs, Inc. (West Grove, PA); Lifespan Biosciences, Inc. (Seattle, WA); Novus Biologicals, LLC (Centennial, CO); RayBiotech Life (Peachtree Corners, GA); Roche Diagnostics (Basel, CH); Rockland Immunochemicals, Inc. (Pottstown, PA); Santa Cruz Biotechnology, Inc. (Dallas, TX); Sigma-Aldrich Corp. (St. Louis, MO); and Thermo Fisher Scientific (Waltham, MA). However, this list is not inclusive, and there are many additional commercial sources of anti-fluorescein antibodies that can be utilized in accordance with the present disclosure. Thus, a person having ordinary skill in the art will clearly and unambiguously be able to identify and select a variety of anti-fluorescein antibodies that can be utilized in accordance with the present disclosure, and as such, no further description of the anti-human Ig antibodies or the characteristics thereof is deemed necessary.

In addition, methods of attaching antibodies to paramagnetic solid phase particles are widely known in the art and well within the purview of a person of ordinary skill in the art. Therefore, no further description thereof is deemed necessary.

Anti-DHEAS monoclonal antibodies are well-known in the art, widely commercially available, and have been vastly studied. For example (but not by way of limitation), a few commercial sources of anti-DHEAS monoclonal antibodies include those available from Antibodies Online, Inc. (Limerick, PA); Creative Diagnostics (Shirley, NY); Lifespan Biosciences, Inc. (Seattle, WA); and MyBioSource, Inc. (San Diego, CA). However, this list is not inclusive, and there are many additional commercial sources of anti-DHEAS monoclonal antibodies that can be utilized in accordance with the present disclosure. Thus, a person having ordinary skill in the art will clearly and unambiguously be able to identify and select a variety of anti-DHEAS monoclonal antibodies that can be utilized in accordance with the present disclosure, and as such, no further description of the anti-human Ig antibodies or the characteristics thereof is deemed necessary.

In addition, methods of labeling monoclonal antibodies with acridinium esters are widely known in the art and well within the purview of a person of ordinary skill in the art. Therefore, no further description thereof is deemed necessary.

In another non-limiting embodiment, the immunoassay kit can include any of the DHEAS-CMO-DMAE conjugates disclosed or otherwise contemplated herein, either alone or in combination with paramagnetic solid phase particles labeled with fluoresceinated anti-DHEAS monoclonal antibody.

The paramagnetic solid phase particles and anti-DHEAS monoclonal antibodies may be any disclosed or otherwise contemplated herein. In addition, methods of labeling monoclonal antibodies with fluorescein are well-known in the art and well within the purview of a person of ordinary skill in the art. Therefore, no further description thereof is deemed necessary.

In addition, the kits of the present disclosure may further contain one or more other component(s) or reagent(s) for performing biological sample collection(s) and/or diagnostic application(s) in accordance with the present disclosure. For example (but not by way of limitation), the kits may include one or more biological sample collection device(s), one or more assay reagent(s), one or more calibration reagent(s), one or more quality control reagent(s), one or more wash reagent(s), etc. The nature of these additional component(s)/reagent(s) will depend upon various factors such as (but not limited to) the type of biological sample and the diagnostic assay format, and identification thereof is well within the skill of one of ordinary skill in the art; therefore, no further description thereof is deemed necessary.

Also, the various components/reagents present in the kit may each be in separate containers/compartments, or various components/reagents can be combined in one or more containers/compartments, depending on the cross-reactivity and stability of the components/reagents. The kit can further include other separately packaged reagents for conducting an assay. In addition, the kit may include a microfluidics device in which the components/reagents are applied.

The relative amounts of the various components/reagents present in the kits can vary widely to provide for concentrations of the components/reagents that substantially optimize the reactions that need to occur during the assay methods and further to optimize substantially the sensitivity of an assay. Under appropriate circumstances one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Positive and/or negative controls may be included with the kit. The kit can further include a set of written instructions explaining how to use the kit. A kit of this nature can be used in any of the methods described or otherwise contemplated herein.

The reagents of the compositions/kits/methods may be provided in any form that allows them to function in accordance with the presently disclosed and claimed inventive concept(s). For example, but not by way of limitation, the reagents may be applied in the form of single aliquot lyophilized reagents. The use of dried reagents in microfluidics devices is described in detail in U.S. Pat. No. 9,244,085, the entire contents of which are hereby expressly incorporated herein by reference.

Certain non-limiting embodiments of the present disclosure are directed to a method of determining a concentration of DHEAS in a biological sample. The method comprises the steps of: (a) combining, either simultaneously or partially or wholly sequentially, to form a mixture: (1) a biological sample suspected of containing DHEAS; (2) any of the dehydroepiandrosterone sulphate-fluorescein (DHEAS-FITC) conjugates disclosed or otherwise contemplated herein; (3) any of the anti-fluorescein paramagnetic solid phase particles disclosed or otherwise contemplated herein; and (4) any acridinium ester-labeled anti-DHEAS monoclonal antibody disclosed or otherwise contemplated herein; and (b) allowing, in the mixture formed in (a), the binding of (2), (3), and (4) to one another and the binding of (4) to DHEAS present in the biological sample. The method may further include the step(s) of: (c) measuring a fluorescence signal generated in the mixture; and/or (d) determining the concentration of DHEAS present in the biological sample based upon an amount of decrease in fluorescence signal observed when compared to a fluorescence signal observed in the absence of biological sample. That is, the method utilizes a competitive format where an amount of fluorescence is inversely proportional to the amount of DHEAS present in the sample.

In certain particular (but non-limiting) embodiments, in step (d) of the method, the concentration of DHEAS present in the biological sample is determined by comparing the fluorescence signal to a calibration curve.

Certain non-limiting embodiments of the present disclosure are directed to a method of determining a concentration of DHEAS in a biological sample. The method comprises the steps of: (a) combining, either simultaneously or partially or wholly sequentially, to form a mixture: (1) a biological sample suspected of containing DHEAS; (2) any of the paramagnetic solid phase particles labeled with fluoresceinated anti-DHEAS monoclonal antibody disclosed or otherwise contemplated herein; and (3) any of the dehydroepiandrosterone sulphate-carboxymethoxylamino-dimethyl acridinium ester (DHEAS-CMO-DMAE) conjugates disclosed or otherwise contemplated herein; and (b) allowing, in the mixture formed in (a), the binding of (3) to (2) or DHEAS present in the biological sample to (2). The method may further include the step(s) of: (c) measuring a fluorescence signal generated in the mixture; and/or (d) determining the concentration of DHEAS present in the biological sample based upon an amount of decrease in fluorescence signal observed when compared to a fluorescence signal observed in the absence of biological sample. In other words, the method utilizes a competitive format where an amount of fluorescence is inversely proportional to the amount of DHEAS present in the sample

In certain particular (but non-limiting) embodiments, in step (d) of the method, the concentration of DHEAS present in the biological sample is determined by comparing the fluorescence signal to a calibration curve.

Certain non-limiting embodiments of the present disclosure are directed to a microfluidics device for determining a concentration of DHEAS in a sample. The microfluidics device includes a compartment capable of receiving a sample suspected of containing DHEAS and one or more of the combinations of assay reagents described or otherwise contemplated herein. For example, but not by way of limitation, the microfluidics device may include (i) a first compartment capable of receiving a sample suspected of containing DHEAS; and one or more of the following: (ii) any of the dehydroepiandrosterone sulphate-fluorescein (DHEAS-FITC) conjugates disclosed or otherwise contemplated herein; (iii) any of the anti-fluorescein paramagnetic solid phase particles disclosed or otherwise contemplated herein; and (iv) any of the acridinium ester-labeled anti-DHEAS monoclonal antibodies disclosed or otherwise contemplated herein. In another non-limiting example, the microfluidics device may include (i) a first compartment capable of receiving a sample suspected of containing DHEAS; and one or both of the following: (ii) paramagnetic solid phase particles labeled with fluoresceinated anti-DHEAS monoclonal antibody; and (iii) a dehydroepiandrosterone sulphate-carboxymethoxylamino-dimethyl acridinium ester (DHEAS-CMO-DMAE) conjugate

Any of the reagents (ii)-(iv) can be disposed in any portion of the microfluidics device that allows the device to function in accordance with the present disclosure. For example (but not by way of limitation), at least one of (ii)-(iv) may be disposed in the first compartment. Alternatively (and/or in addition thereto), the microfluidics device may include at least a second compartment that is capable of being in fluidic communication with the first compartment, and one or more of (ii)-(iv) may be disposed in the second compartment. In addition, the microfluidics device may include other additional compartments, and the reagents (ii)-(iv) can be split between three different compartments, if desired.

The microfluidics devices of the present disclosure may include a sample application chamber in which a sample may be applied and an inlet channel in fluidic communication therewith that is also in fluidic communication with one or more compartments containing one or more of the reagents described herein above. The device may be provided with any number of compartments, any arrangement of compartments, and any distribution of the components there between, so long as the device is able to function in accordance with the present disclosure.

Any of the compartments of the microfluidics device may be sealed to maintain reagent(s) applied therein in a substantially air tight environment until use thereof; for example, compartments containing lyophilized reagent(s) may be sealed to prevent any unintentional reconstitution of the reagent. The inlet channel and a compartment, as well as two compartments, may be described as being “capable of being in fluidic communication” with one another; this phrase indicates that the compartment(s) may still be sealed, but the two compartments are capable of having fluid flow there between upon puncture of a seal formed therein or there between.

The microfluidics devices of the present disclosure may be provided with any other desired features known in the art or otherwise contemplated herein. For example, but not by way of limitation, the microfluidics device may further include a read chamber; the read chamber may be the compartment containing the paramagnetic solid phase particles, or the read chamber may be in fluidic communication with said compartment. The microfluidics device may further include one or more compartments containing other solutions, such as but not limited to, wash solutions, dilution solutions, excipients, interference solutions, positive controls, negative controls, quality controls, and the like. For example, the microfluidics device may include one or more compartments containing a wash solution, and these compartment(s) may be capable of being in fluidic communication with any other compartment(s) of the device. In another example, the microfluidics device may further include one or more compartments containing at least one excipient for dissolution of one or more dried reagents, and the compartment(s) may be capable of being in fluidic communication with any other compartment(s) of the device. Further, the microfluidics device may further include one or more compartments containing a dilution solution, and the compartment(s) may be capable of being in fluidic communication with any other compartment(s) of the device.

In addition, any of the kits/microfluidics devices described or otherwise contemplated herein may include multiple assays multiplexed in a single kit/device.

### Example

An Example is provided hereinbelow. However, the present disclosure is to be understood to not be limited in its application to the specific experimentation, results, and laboratory procedures disclosed herein after. Rather, the Example is simply provided as one of various embodiments and is meant to be exemplary, not exhaustive.

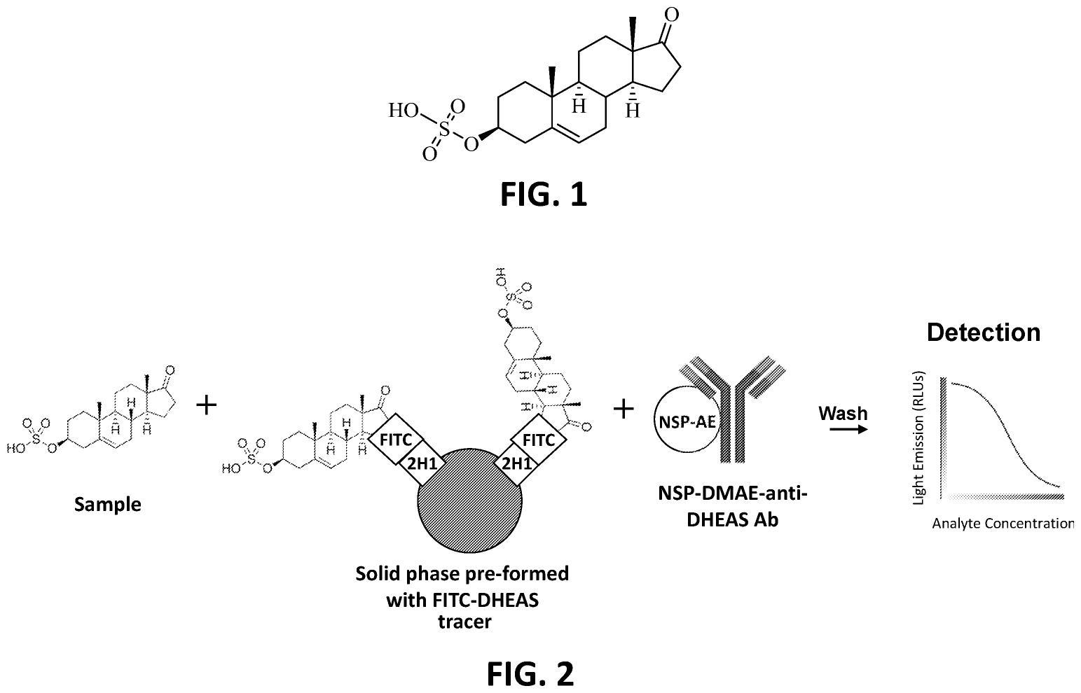

DHEAS (FIG. 1) is an adrenal steroid, and abnormal levels of DHEAS are known in the art to be associated with many different disorders and conditions, as outlined above in the Background section. Methods for the measurement of DHEAS are desired in order to provide a mechanism for the assessment of DHEAS levels in human patients. However, immunoassays for the detection of DHEAS have encountered issues with assay sensitivity, ambient temperature effect (ATE), and susceptibility to biotin interference.

In response to the disadvantages and deficiencies of the currently available DHEAS assays, a series of novel DHEAS derivatives and conjugates were designed and synthesized for use in an immunoassay platform based on acridinium ester (AE) technology (Natrajan et al. (2010); Natrajan et al. (2011); and Natrajan et al. (2012)). These novel DHEAS derivatives and conjugates included the dehydroepiandrosterone sulphate-fluorescein (DHEAS-FITC) and dehydroepiandrosterone sulphate-acridinium ester (DHEAS-AE) conjugates. These DHEAS-FITC and DHEAS-AE conjugates were evaluated on Atellica/ADVIA Centaur immunoanalyzer (Siemens Healthcare Diagnostics Inc., Tarrytown, NY) using a competitive assay format. The DHEAS-CMO-EDA-FITC (8) compound (DHEAS-carboxymethoxylamino-ethyldiamine-fluorescein; paired with anti-FITC labeled paramagnetic solid phase particles (anti-FITC-PMP)), demonstrated excellent assay performance relative to assay requirements and has been selected as one of the primary candidate reagents for the DHEAS II immunoassay.

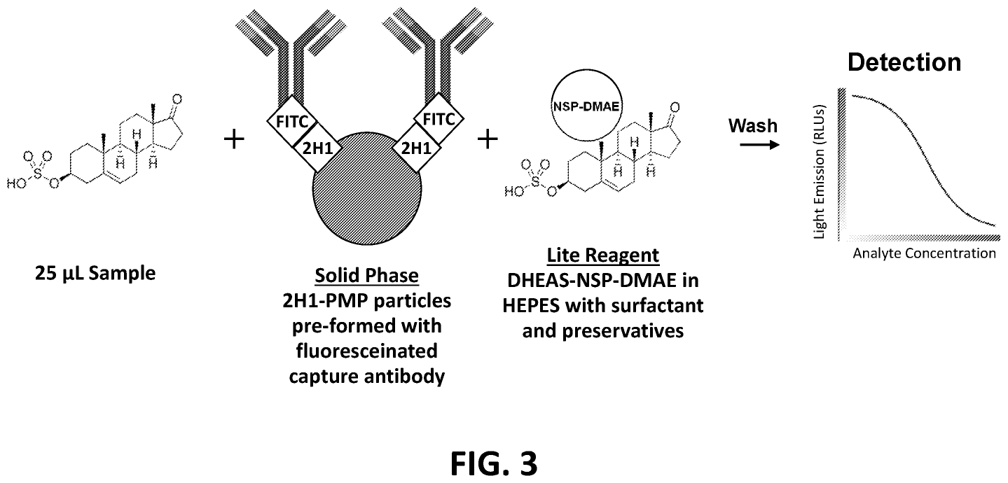

Reagent Design: The Atellica/ADVIA Centaur DHEAS reagents included a solid phase, an ancillary reagent, and a lite reagent. The current commercial assay uses a DHEAS-NSP-AE (lite reagent) paired with biotinylated mouse monoclonal antibody (mAb, ancillary reagent) and streptavidin-coated solid phase. In the assays of the present disclosure, anti-FITC-PMP particles were selected for the solid phase instead of streptavidin-coated particles to prevent biotin interference. Two assay formats were explored for this immunoassay (FIGS. 2-3). In Format 1 (FIG. 2), the solid phase reagent contains magnetic particles labeled with fluoresceinated DHEAS, and the lite reagent contains the mAb-AE conjugate. Lite reagent antibody associates with the DHEAS-FITC on the solid phase. Endogenous DHEAS competes with DHEAS-FITC-bound solid phase for the mAb-AE in the lite reagent, creating an inverse relationship between RLU signal strength and endogenous analyte concentration when measured against a calibration curve.

Both DHEAS-FITC and mAb-AE reagents needed to be newly developed and evaluated to support the assay Format 1 (FIG. 2). AE-labelled monoclonal antibodies were prepared by methods known and characterized in the art; however, DHEAS-FITC conjugates and methods of synthesis thereof were not known prior to the present disclosure, and as such, their performance in a DHEAS II immunoassay was not considered prior to the present disclosure.

A second DHEAS II format was also investigated—this assay format as referred to as Format 2 and depicted in FIG. 3. In Format 2, the solid phase reagent contains magnetic particles labeled with fluoresceinated mAb. The lite reagent contains a DHEAS-AE tracer. Format 2 operates using the same competitive principal as Format 1. An addition of endogenous DHEAS from a patient sample to the reaction disrupts the binding of the DHEAS-AE tracer to the solid phase, resulting in decreased signal generation. In Format 2, design and development focused on the synthesis of different DHEAS-AE tracers paired with the fluoresceinated mAb-coated PMP solid phase with the aim of improving the assay performance while eliminating biotin interference.

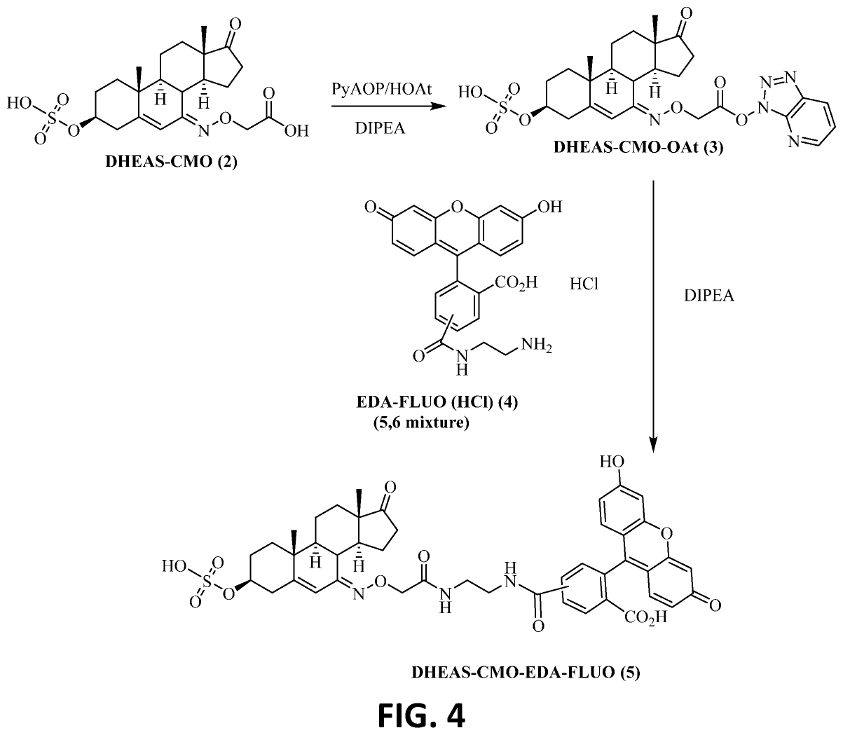

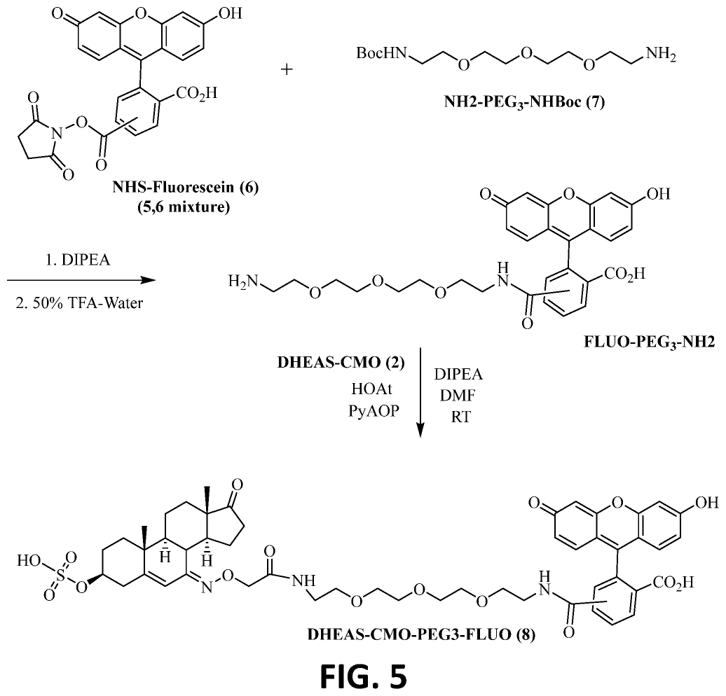

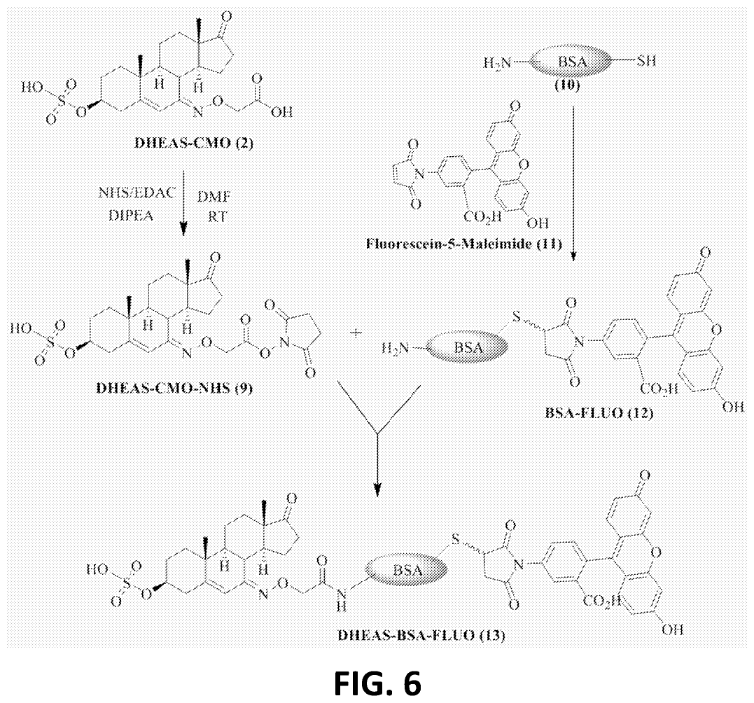

For Format 1 (FIG. 2), DHEAS-CMO-EDA-Fluorescein (5) was designed and synthesized by reacting DHEAS-CMO with EDA-Fluorescein for initial assay studies (FIG. 4). Early data indicated that this compound yielded promising functional data, significantly reducing the ambient temperature effect (ATE) compared to the currently available commercial assay. DHEAS-CMO-PEG3-Fluorescein (8) was also prepared to evaluate whether alternative hydrophilic linker structures confer additional benefits to the assay performance (FIG. 5). In addition, BSA was utilized as protein linker which can accommodate multiple stochiometries of DHEAS and fluorescein moieties on BSA to prepare DHEAS-BSA-fluorescein conjugate (FIG. 6). The detailed assay performance of these three reagents are discussed below.

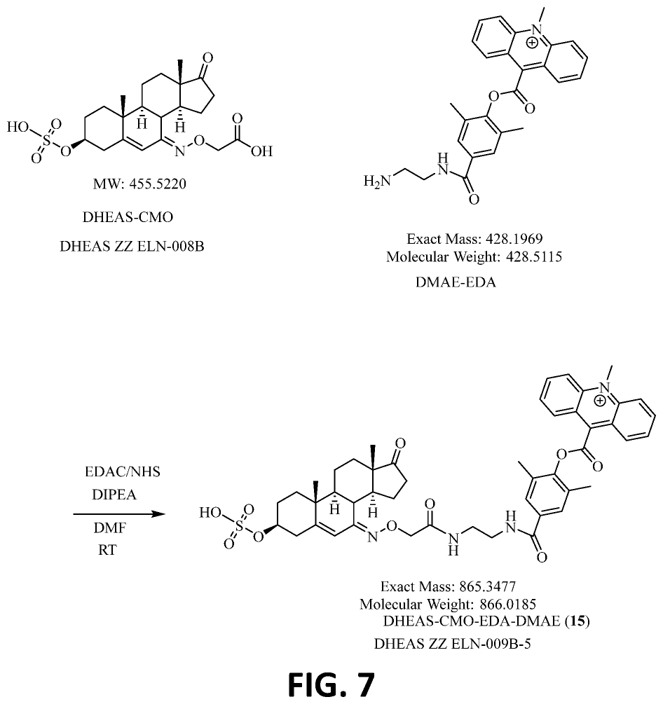

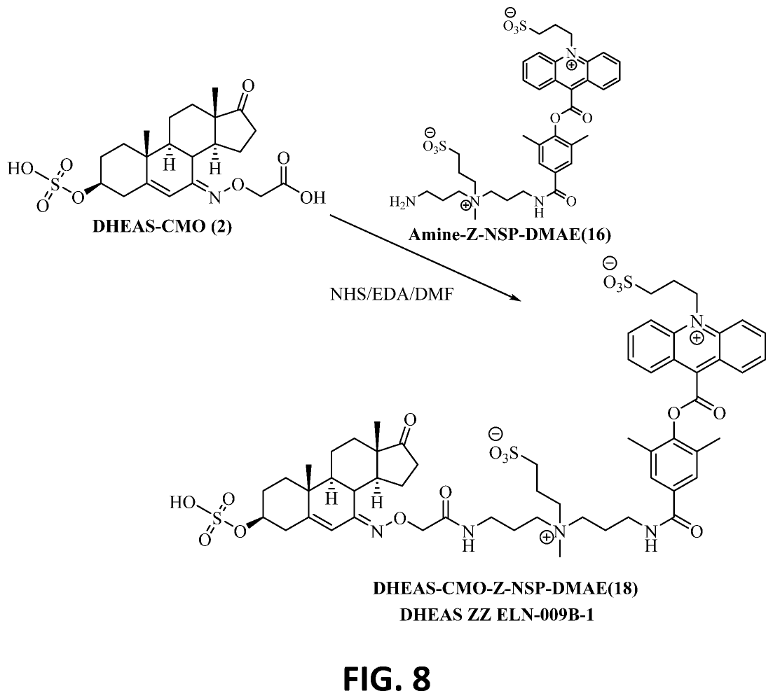

For assay Format 2 (FIG. 3), DHEAS-CMO was selected for conjugation to a DMAE acridinium ester for initial characterization studies. Early data demonstrated that using this conjugate, DHEAS-CMO-EDA-DMAE (15), produced a promising standard curve shape and significantly reduced ambient temperature bias. Given this initial performance, a conjugate with Z-NSP-DMAE at the same linkage position was also prepared to determine if the differing AE structure would confer any additional ATE benefit to the assay. The performance of both DHEAS-AEs (15, 18) are discussed below. The syntheses of both DHEAS-AEs are shown in the schemes in FIGS. 7-8, respectively.

The evaluation of both the DHEAS-FITC and DHEAS-AE conjugates was carried out using the ADVIA Centaur family of immunoassay analyzers at Siemens Healthcare Diagnostics Inc., Tarrytown, NY.

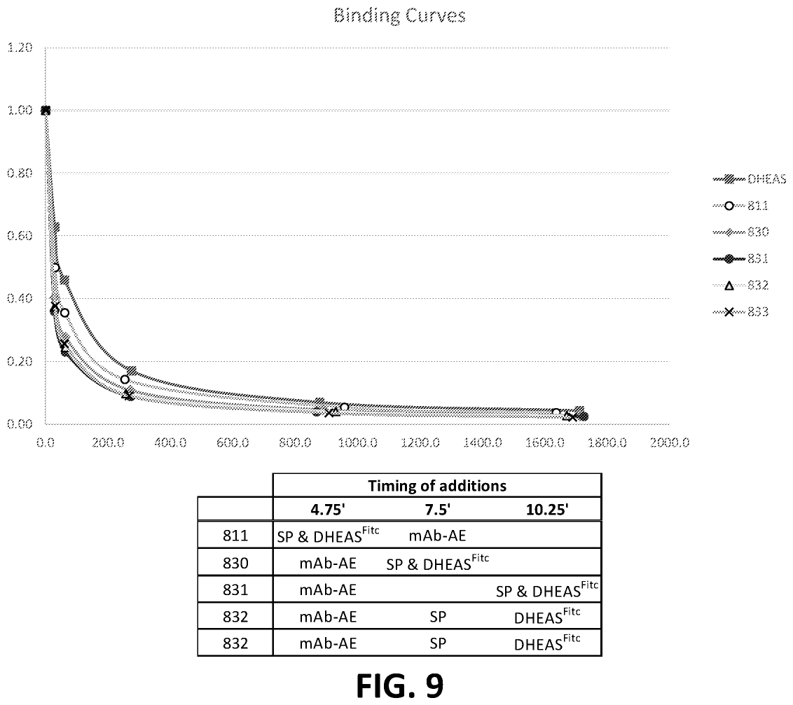

Functional performance (binding curve shape, precision estimates, and ambient temperature bias (ATE)) of fluoresceinated conjugates DHEAS-CMO-EDA-FITC, DHEAS-CMO-PEG3-FITC, and DHEAS-BSA-FITC ((5), (8), (13) respectively) were evaluated in DHEAS II immunoassay Format 1. The conjugates DHEAS-CMO-PEG3-FITC and DHEAS-BSA-FITC showed minimal improvement compared to the currently available commercial assay (data not shown). DHEAS-CMO-EDA-FITC conjugate (5) demonstrated improved binding profile (FIG. 9) with a comparable or better sensitivity compared to the currently available commercial assay. More importantly, as shown in Tables 1-2, assay Format 1 with conjugate (5) minimized ATE to ≤10%, greatly improving assay performance compared the commercial assay (% ATE around 29%).

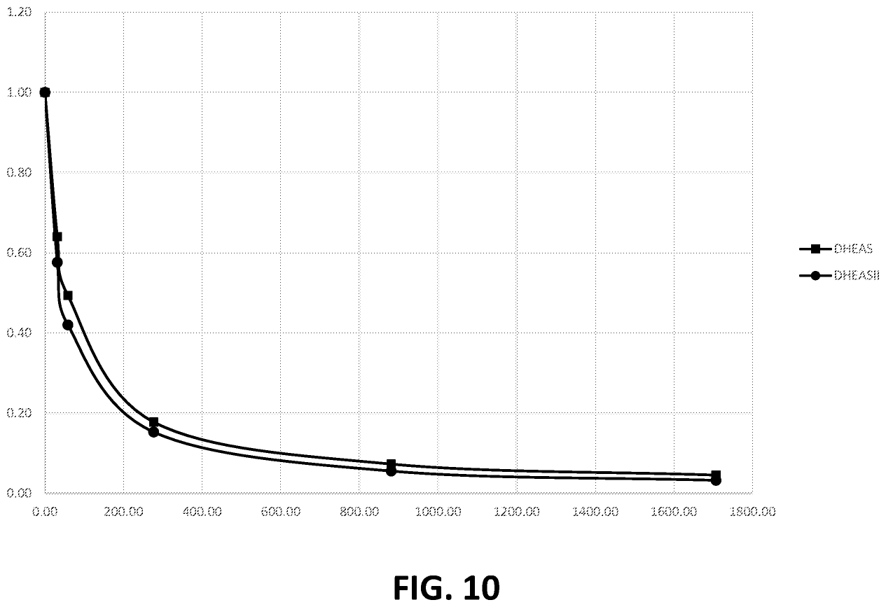

The two DHEAS-AE conjugates (15, 18) were also evaluated in assay Format 2 using a prototype ADVIA Centaur DHEAS II immunoassay. The two AE conjugates demonstrated promising assay performance compared to the commercial assay (FIG. 10). ATE estimates for both AEs are better than of the commercial assay (Table 3).

Based on overall preliminary assay performance, conjugate (5) has the best ATE and assay sensitivity among the reagents tested.

TABLE 1
Ambient Temperature Performance of Current Commercial DHEAS Assay

| | Commercial DHEAS Kit RLUs | | | | Commercial DHEAS Kit Dose | | | |
|---|---|---|---|---|---|---|---|---|
| Sample | 18° C. | 24° C. | 30° C. | % Bias | 18° C. | 24° C. | 30° C. | % Bias |
| BR40361 | 210467 | 236479 | 257415 | 20% | 93.7 | 77.0 | 66.2 | −36% |
| BR40362 | 135934 | 156179 | 167797 | 20% | 177.2 | 146.2 | 131.9 | −31% |
| BR40363 | 52662 | 59711 | 65703 | 22% | 611.3 | 518.1 | 457.7 | −30% |
| BRH1628711 | 268454 | 291782 | 308113 | 14% | 61.3 | 51.9 | 46.3 | −29% |
| BRH1628719 | 153425 | 167026 | 180280 | 16% | 149.9 | 132.7 | 118.7 | −23% |
| BRH1628727 | 99026 | 110661 | 119950 | 19% | 269.9 | 233.4 | 209.8 | −26% |
| BRH1628728 | 48135 | 52754 | 56972 | 17% | 689.9 | 610.4 | 551.1 | −23% |
| BRH1628744 | 247660 | 272525 | 292956 | 17% | 71.0 | 59.5 | 51.5 | −33% |
| Mean % Bias | | | | 18% | | | | −29% |

TABLE 2
Ambient Temperature Performance of New DHEAS
II Assay (FIG. 2) Using DHEAS-CMO-EDA-FITC

| | DHEASII Kit RLUs | | | | Commercial DHEAS Kit Dose | | | |
|---|---|---|---|---|---|---|---|---|
| Sample | 18° C. | 24° C. | 30° C. | % Bias | 18° C. | 24° C. | 30° C. | % Bias |
| BR40361 | 460398 | 381438 | 432676 | −7% | 106.3 | 108.0 | 121.3 | 14% |
| BR40362 | 314170 | 262649 | 309646 | −2% | 221.2 | 221.2 | 226.9 | 3% |
| BR40363 | 148803 | 125387 | 140171 | −7% | 737.4 | 724.6 | 810.1 | 10% |
| BRH1628711 | 504724 | 429489 | 490384 | −3% | 86.5 | 83.1 | 92.3 | 7% |
| BRH1628719 | 325003 | 278490 | 326079 | 0% | 208.5 | 198.5 | 207.4 | −1% |
| BRH1628727 | 243884 | 205181 | 241086 | −1% | 338.0 | 328.5 | 344.5 | 2% |
| BRH1628728 | 145570 | 124681 | 142708 | −2% | 763.3 | 730.7 | 787.7 | 3% |
| BRH1628744 | 478153 | 408355 | 477405 | 0% | 97.8 | 92.1 | 98.1 | 0% |
| Mean % Bias | | | | −3% | | | | 5% |

TABLE 3
Ambient Temperature Performance of New DHEAS II Assay
(FIG. 3) Using DHEAS-CMO-EDA-DMAE Conjugate and
DHEAS-CMO-Z-DMAE versus Commercial DHEAS Assay

| | DHEAS-CMO-EDA-DMAE RLUs | | | | | | |
|---|---|---|---|---|---|---|---|
| | 18° C. | | 24° C. | | 30° C. | | % Rec |
| BR40361 | 1252972 | 1.8% | 1287708 | 0.5% | 1247291 | 1.3% | 100% |
| BR40362 | 775002 | 1.8% | 776174 | 3.0% | 747334 | 2.9% | 96% |
| BR40363 | 264134 | 1.7% | 273114 | 1.4% | 270112 | 1.1% | 102% |
| BRH1628711 | 1509032 | 2.7% | 1478153 | 0.9% | 1422583 | 2.3% | 94% |
| BRH1628719 | 785206 | 2.1% | 801677 | 2.4% | 767314 | 2.2% | 98% |
| BRH1628727 | 527225 | 3.3% | 521796 | 1.2% | 517882 | 4.1% | 98% |
| BRH1628728 | 247846 | 1.2% | 251916 | 3.4% | 241386 | 3.3% | 97% |
| BRH1628744 | 1402476 | 0.8% | 1394687 | 4.3% | 1426526 | 0.4% | 102% |
| | | | | | | | 98% |

| | DHEAS-CMO-EDA-DMAE Dose | | | | | | |
|---|---|---|---|---|---|---|---|
| | 18° C. | | 24° C. | | 30° C. | | % Bias |
| BR40361 | 103.21 | 2.6% | 99.12 | 0.8% | 103.88 | 1.9% | 0.7% |
| BR40362 | 197.63 | 2.3% | 197.36 | 3.9% | 207.08 | 3.7% | 4.8% |
| BR40363 | 746.87 | 2.1% | 715.93 | 1.8% | 725.92 | 1.4% | −2.9% |
| BRH1628711 | 77.77 | 4.3% | 80.31 | 1.5% | 85.31 | 3.6% | 9.4% |
| BRH1628719 | 194.38 | 2.6% | 189.31 | 3.2% | 200.19 | 2.9% | 3.1% |
| BRH1628727 | 319.38 | 4.0% | 323.18 | 1.5% | 326.62 | 5.1% | 2.2% |
| BRH1628728 | 809.78 | 1.6% | 793.91 | 4.3% | 838.49 | 4.1% | 3.6% |
| BRH1628744 | 87.16 | 1.2% | 88.08 | 6.7% | 84.90 | 0.6% | −2.6% |
| | | | | | | | 2.3% |

In summary, a series of DHEAS-FITC and DHEAS-AE conjugates have been successfully designed, synthesized, and evaluated for use in DHEAS II immunoassays (Formats 1-2, FIGS. 2-3, respectively). Based on overall assay performance, SP phase reagents (Format 1) derived from DHEAS-CMO-EDA-Fluorescein (5) (FIG. 4) have promising low-end assay sensitivity and the best ambient temperature effect (ATE) on the assay in comparison to that of the current commercial assay. Format 1 employs the 2H1-anti-FITC PMP solid phase that minimizes the risk of biotin interference. DHEAS-CMO-EDA-Fluorescein was selected as an excellent candidate meeting preliminary assay requirements (Tables 2 and 3). The conjugate is an important component for the development of a commercial ATELLICA/ADVIA Centaur DHEAS II immunoassay.

Thus, in accordance with the present disclosure, there have been provided compositions, devices, and kits, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
1. Haning R V. “Using DHEAS to monitor androgen disorders.” Contemp Ob/Gyn. 1981; 18 (9): 117-132.
2. Kasick J M, Bergfeld W F, Steck W D, Gupta M K. “Adrenal androgenic female-pattern alopecia: sex hormones and the balding woman.” Cleve Clin Q. 1983; 50 (2): 111-122.
3. Cohen H N, Wallace A M, Beastall G H, Fogelman I, Thomson J A. “Clinical value of adrenal androgen measurement in the diagnosis of delayed puberty.” Lancet. 1981; 1 (8222): 689-692.
4. Braunstein G D. “Coping with hirsutism-management strategies.” Contemp Ob/Gyn. 1981; 17 (5): 189-193.
5. de Peretti E, Forest M G. “Pattern of plasma dehydroepiandrosterone sulfate levels in humans from birth to adulthood: evidence for testicular production.” J Clin Endocrinol Metab. 1978; 47 (3): 572-577.
6. Zappulla F, Ventura D, Capelli M, et al. “Gonadal and adrenal secretion of dehydroepiandrosterone sulfate in prepubertal and pubertal subjects.” J Endocrinol Invest. 1981; 4 (2): 197-202.
7. Vermeulen A. “Androgen secretion by adrenals and gonads.” Mahesh V, Greenblatt R B, eds. Hirsutism and Virilism. Boston: John Wright-PSG; 1983:17-34.
8. Sciarra F. “Diagnosis of virilizing syndromes: endocrinological parameters.” Molinatti G, et al, eds. Androgenization in Women. New York: Raven Press; 1983:85-113.
9. Haning R V Jr, Carlson I H, Shapiro S S, Nolten W E. “Testosterone free index correlates best with dehydroepiandrosterone sulfate.” Fertil Steril. 1981; 36 (6): 757-765.
10. Lobo R A, Paul W L, Goebelsmann U. “Serum levels of DHEAS in gynecologic endocrinopathy and infertility.” Obstet Gynecol. 1981; 57 (5): 607-612.
11. Buvat J, Dewailly D, Marcolin G, Buvat-Herbaut M, Racadot A, Fossati P. “Investigative strategy of hyperandrogenism in women.” Horm Res. 1983; 18 (1-3): 106-116.
12. Natrajan, A.; Sharpe, D.; Costello, J.; Jiang, Q. P. “Enhanced Immunoassay Sensitivity Using Chemiluminescent Acridinium Esters with Increased Light Output” Ann Biochem 2010, vol. 406, 204.
13. Natrajan, A.; Sharpe, D.; Wen, D. “Effect of Surfactants On the Chemiluminescence of Acridinium Dimethylphenyl Ester Labels and Their Conjugates” Org Biomol Chem 2011, vol. 9, 5092.
14. Natrajan, A.; Sharpe, D.; Wen, D. “Zwitterionic Reagents for Labeling, Cross-Linking and Improving the Performance of Chemiluminescent Immunoassays” Org Biomol Chem 2012, vol. 10, 188

What is claimed is:

1. An immunoassay reagent, comprising:
a dehydroepiandrosterone sulphate-fluorescein (DHEAS-FITC) conjugate.

2. The immunoassay reagent of claim 1, wherein the DHEAS-FITC conjugate comprises DHEAS-CMO-EDA-FITC (DHEAS-carboxymethoxylamino-ethyldiamine-fluorescein).

3. The immunoassay reagent of claim 1, wherein the DHEAS-FITC conjugate comprises DHEAS-CMO-PEG3-Fluorescein (DHEAS-carboxymethoxylamino-(polyethylene glycol)₃-Fluorescein) or DHEAS-CMO-BSA-Fluorescein (DHEAS-carboxymethoxylamino-bovine serum albumin-Fluorescein).

4. The immunoassay reagent of claim 1, wherein the fluorescein is fluorescein isothiocyanate (FITC).

5. An immunoassay reagent, comprising:
a dehydroepiandrosterone sulphate-carboxymethoxylamino-dimethyl acridinium ester (DHEAS-CMO-DMAE) conjugate.

6. The immunoassay reagent of claim 5, wherein the DHEAS-AE conjugate comprises DHEAS-CMO-EDA-DMAE (DHEAS-carboxymethoxylamino-ethyldiamine-dimethyl acridinium ester) or DHEAS-CMO-Z-NSP-DMAE (DHEAS-carboxymethoxylamino-Z-N-sulfopropyl-dimethyl acridinium ester).

7. An immunoassay kit, comprising:
a dehydroepiandrosterone sulphate-fluorescein (DHEAS-FITC) conjugate;
anti-fluorescein paramagnetic solid phase particles; and
an acridinium ester-labeled anti-DHEAS monoclonal antibody.

8. The immunoassay kit of claim 7, wherein the DHEAS-FITC conjugate comprises DHEAS-CMO-EDA-FITC (DHEAS-carboxymethoxylamino-ethyldiamine-fluorescein).

9. The immunoassay kit of claim 7, wherein the fluorescein of the DHEAS-FITC conjugate is fluorescein isothiocyanate (FITC).

10. The immunoassay kit of claim 9, wherein the DHEAS-FITC conjugate comprises DHEAS-CMO-PEG3-Fluorescein (DHEAS-carboxymethoxylamino-(polyethylene glycol)₃-Fluorescein) or DHEAS-CMO-BSA-Fluorescein (DHEAS-carboxymethoxylamino-bovine serum albumin-Fluorescein).

11. An immunoassay kit, comprising:
paramagnetic solid phase particles labeled with fluoresceinated anti-DHEAS monoclonal antibody; and
a dehydroepiandrosterone sulphate-carboxymethoxylamino-dimethyl acridinium ester (DHEAS-CMO-DMAE) conjugate.

12. The immunoassay kit of claim 11, wherein the DHEAS-AE conjugate comprises DHEAS-CMO-EDA-DMAE (DHEAS-carboxymethoxylamino-ethyldiamine-dimethyl acridinium ester) or DHEAS-CMO-Z-NSP-DMAE (DHEAS-carboxymethoxylamino-Z-N-sulfopropyl-dimethyl acridinium ester).

13. A method of determining a concentration of DHEAS in a biological sample, the method comprising the steps of:
(a) combining, either simultaneously or partially or wholly sequentially, to form a mixture:
(1) a biological sample suspected of containing DHEAS;
(2) a dehydroepiandrosterone sulphate-fluorescein (DHEAS-FITC) conjugate;
(3) anti-fluorescein paramagnetic solid phase particles; and
(4) an acridinium ester-labeled anti-DHEAS monoclonal antibody;
(b) allowing, in the mixture formed in (a), the binding of (2), (3), and (4) to one another and the binding of (4) to DHEAS present in the biological sample; and
(c) measuring a fluorescence signal generated in the mixture; and
(d) determining the concentration of DHEAS present in the biological sample based upon an amount of decrease in fluorescence signal observed when compared to a fluorescence signal observed in the absence of biological sample.

14. The method of claim 13, wherein the DHEAS-FITC conjugate comprises DHEAS-CMO-EDA-FITC (DHEAS-carboxymethoxylamino-ethyldiamine-fluorescein).

15. The method of claim 14, wherein the fluorescein of the DHEAS-FITC conjugate is fluorescein isothiocyanate (FITC).

16. The method of claim 14, wherein the DHEAS-FITC conjugate comprises DHEAS-CMO-PEG3-Fluorescein (DHEAS-carboxymethoxylamino-(polyethylene glycol)₃-Fluorescein) or DHEAS-CMO-BSA-Fluorescein (DHEAS-carboxymethoxylamino-bovine serum albumin-Fluorescein).

17. The method of claim 14 wherein, in step (d), the concentration of DHEAS present in the biological sample is determined by comparing the fluorescence signal to a calibration curve.

18. A method of determining a concentration of DHEAS in a biological sample, the method comprising the steps of:
(a) combining, either simultaneously or partially or wholly sequentially, to form a mixture:
(1) a biological sample suspected of containing DHEAS;
(2) paramagnetic solid phase particles labeled with fluoresceinated anti-DHEAS monoclonal antibody; and
(3) a dehydroepiandrosterone sulphate-carboxymethoxylamino-dimethyl acridinium ester (DHEAS-CMO-DMAE) conjugate;
(b) allowing, in the mixture formed in (a), the binding of (3) to (2) or DHEAS present in the biological sample to (2); and
(c) measuring a fluorescence signal generated in the mixture; and
(d) determining the concentration of DHEAS present in the biological sample based upon an amount of decrease in fluorescence signal observed when compared to a fluorescence signal observed in the absence of biological sample.

19. The method of claim 18, wherein the DHEAS-AE conjugate comprises DHEAS-CMO-EDA-DMAE (DHEAS-carboxymethoxylamino-ethyldiamine-dimethyl acridinium ester) or DHEAS-CMO-Z-NSP-DMAE (DHEAS-carboxymethoxylamino-Z-N-sulfopropyl-dimethyl acridinium ester).

20. The method of claim 18 wherein, in step (d), the concentration of DHEAS present in the biological sample is determined by comparing the fluorescence signal to a calibration curve.

21. A microfluidics device for determining a concentration of DHEAS in a sample, the microfluidics device comprising:
(i) a first compartment capable of receiving a sample suspected of containing DHEAS;
(ii) a dehydroepiandrosterone sulphate-fluorescein (DHEAS-FITC) conjugate;
(iii) anti-fluorescein paramagnetic solid phase particles; and
(iv) an acridinium ester-labeled anti-DHEAS monoclonal antibody.

22. The microfluidics device of claim 21, wherein at least one of (ii)-(iv) is disposed in the first compartment.

23. The microfluidics device of claim 21, further defined as comprising at least a second compartment that is capable of being in fluidic communication with the first compartment, wherein at least one of (ii)-(iv) is disposed in the second compartment.
